(19) United States
(12) Patent Application Publication
(10) Pub. No.: US 2018/0321236 A1
(43) Pub. Date: Nov. 8, 2018

(54) CORE ELEMENTS FOR POINT OF CARE DIAGNOSIS OF TUBERCULOSIS

(71) Applicants: UNIVERSITY OF PRETORIA, Preetoria (ZA); CSIR, Pretoria (ZA)

(72) Inventors: Jan Adrianus Verschoor, Die Wilgers (ZA); Ikechukwu Okeke, Pretoria (ZA); Lonji Kalombo, Pretoria (ZA); Yolandy Lemmer, Krugersdorp (ZA)

(21) Appl. No.: 15/773,889

(22) PCT Filed: Nov. 4, 2016
(86) PCT No.: PCT/IB2016/056643

§ 371 Date: May 4, 2018

(30) Foreign Application Priority Data
Nov. 6, 2015 (ZA) 2015/08200

(51) Int. Cl. G01N 33/569; G01N 33/543

(52) U.S. Cl. CPC G01N 33/5695; G01N 33/54346; G01N 2469/20; G01N 2333/35; G01N 2800/12; G01N 33/5438

(57) ABSTRACT

The invention provides a method of diagnosing tuberculosis by using a novel antibody biomarker capturing vehicle, activating a screen-printed electrode using chemical and mechanical polishing, and immobilising mycolic acid antigen biomarkers on the activated electrode. The novel antibody biomarker capturing vehicle is produced by introducing isolated mycolic acid antigens of tuberculous mycobacterial origin onto an outer surface of a nanoparticle such that the mycolic acid antigens are presented as antibody biomarker capturing agents. The solvent resistant screen-printed electrode is activated by both chemically and mechanically polishing the electrode. The mycolic acid antigens are immobilised on the activated electrode by incubating the activated electrode with a mycolic acid-dimethylformamide solution to allow mycolic acid antigens to adsorb onto the activated electrode.

This invention relates to a method of diagnosing tuberculosis. It relates in particular to a method of diagnosing tuberculosis by using a novel antibody biomarker capturing vehicle, activating a screen-printed electrode using chemical and mechanical polishing, and immobilising mycolic acid antigen biomarkers on the activated electrode.

According to the World Health Organization (WHO), 8.6 million people were infected with tuberculosis (TB) in 2012 and 1.3 million people died as a result thereof. Of the 1.3 million TB-related deaths, 20% were co-infected with Human Immunodeficiency Virus (HIV) and about 75% of these cases were from Africa. In Africa, TB is often the first sign of HIV infection and is the major cause of death amongst HIV-infected patients. The highest prevalence of HIV is found in Southern Africa which also has the highest incidence of TB. Difficulty in accurately and effectively diagnosing TB has played a major role in hampering the elimination of the disease. It is still a challenge for current diagnostic tests to accurately detect early TB in a human. Misdiagnosis and late-diagnosis of TB contributes a role in an increased mortality amongst infected patients. In developing countries, one of the major barriers that affects the development and implementation of new TB diagnostics is the high cost thereof.

The Mycolic acid Antibodies Real-Time Inhibition (MARTI) test has the ability to accurately detect low affinity patient anti-mycolic acids antibodies as biomarkers for active TB. The MARTI test was patented in 2005 by the University of Pretoria (U.S. Pat. No. 7,851,166). Initially the validation of the MARTI test was done on an IAsys waveguide affinity biosensor. This was not a user-friendly or economic technology. In a later developed format, the MARTI test was used with an ESPRIT surface plasmon resonance (SPR) biosensor. Both the ESPRIT and IAsys biosensors are evanescent field mass-detecting devices, which make use of a cuvette system rather than a flow cell. A drawback to performing the MARTI test on an SPR biosensor is its heavy instrumentation and cost of maintenance associated with SPR.

The standard ELISA immunoassay is an ineffective TB diagnostic tool due to its inherent property of registering the binding of only the highest affinity antibodies to antigen in a serum. This is because of the washing steps required in ELISA, which remove the low affinity antibodies. In comparison to the ELISA assay, the MARTI test has an increased sensitivity and specificity because it does not require a washing step after sample contact with the immobilized mycolic acid antigen. A major advantage of the MARTI test is therefore that it can sensitively detect low affinity antibodies, making it a more accurate diagnostic test.

Electrical impedance spectroscopy (EIS) is more suitable than SPR evanescent field lo devices as transduction technology for antibody binding detection in MARTI, since it requires no heavy instrumentation. Signal processing can nowadays be done by means of a hand-held, battery operated potentiostat. Such potentiostats are essentially service-free, unlike SPR which requires expensive annual maintenance. A point-of-care TB diagnostic device must be affordable, accurate, be simple to use, require minimal amounts of biological sample, be sensitive and specific, be easy to read, be able to diagnose rapidly (at least 20 tests per day) and be able to generate same day results. With affordable, disposable electrodes, the MARTI test on EIS holds the potential to fulfil these requirements.

Mycolic acids (MA) are the dominant lipids found in the outer cell wall of Myobacterium species, and have been shown to play a key role in the pathogen's virulence and to be immunogenic. MA antigens have been immobilized from hexane solution onto solvent-resistant screen-printed electrodes (SPEs) coated with octadecanethiol (ODT). This method of antigen immobilization is, however, poorly reproducible and wasteful on electrodes and MA. It was therefore necessary to identify a more reproducible and affordable protocol of MA antigen immobilization on solvent resistant electrodes. The current invention addresses this problem.

The current invention further provides a stable suspended nanoparticle incorporating MA for presentation for antibody binding inhibition. This addresses an existing problem of an unstable liposome carrier experienced with MARTI. The current nanoparticle carrier is stable to oxidation, has an increased shelf life, and is easily suspendible thereby improving on the MARTI test.

According to a first aspect of the invention, there is provided a method of making an antibody biomarker capturing vehicle, the method including:
introducing isolated mycolic acid antigens of tuberculous mycobacterial origin onto an outer surface of a nanoparticle to obtain a mycolic acid antigen-containing nanoparticle wherein mycolic acid antigens are presented as antibody biomarker capture agents.

The nanoparticle may have a size of 0.2 μm or less.

The nanoparticle may be of a poly(lactic-co-glycolic acid).

According to a second aspect of the invention, there is provided an antibody biomarker capturing vehicle suitable for use in diagnosing tuberculosis, the antibody biomarker capturing vehicle comprising a nanoparticle having isolated mycolic acid of tuberculous mycobacterial origin present as a biomarker capturing agent on an outer surface thereof.

According to a third aspect of the invention, there is provided a method of activating is a solvent resistant screen-printed electrode, suitable for use in diagnosing tuberculosis, the method including both chemically and mechanically polishing the electrode, thereby to obtain an activated solvent resistant screen-printed electrode.

The electrode may be a gold, solvent resistant, screen-printed electrode. The electrode may be a disposable electrode.

Piranha acid may be used for the chemical polishing of the electrode. Alumina may be used for the mechanical polishing of the electrode.

The chemical polishing may be effected first, whereafter the mechanical polishing is effected.

The invention extends to an activated screen-printed electrode when activated by the method of the third aspect of the invention.

According to a fourth aspect of the invention, there is provided a method of immobilising mycolic acid antigens on an activated screen-printed electrode surface suitable for use in diagnosing tuberculosis, the method including:
dissolving mycolic acid of tuberculous mycobacterial origin in dimethylformamide to form a mycolic acid-dimethylformamide solution; and
incubating an activated screen-printed electrode with the mycolic acid-dimethylformamide solution, to allow mycolic acid antigens to adsorb onto the activated electrode, to produce a screen-printed electrode containing immobilized mycolic acid antigens.

The electrode may be a gold screen-printed electrode. The electrode may be solvent resistant. The electrode may be a disposable electrode.

The electrode may be washed following the incubation period. The incubation period may be about one hour. The electrode may be washed in deionised water.

The activated screen-printed electrode may be that obtained by the method of the third aspect of the invention.

According to a fifth aspect of the invention, there is provided a method of diagnosing tuberculosis, which includes
using a mycolic acid antibodies real-time inhibition test, employing electrochemical impedance spectroscopy, to diagnose the presence of active tuberculosis in a sample from a patient suspected of having active tuberculosis;
using the antibody biomarker capturing vehicle according to the second aspect of the invention; and/or
using an activated solvent resistant screen-printed electrode obtained by the method of the third aspect of the invention; and/or
using the screen-printed electrode containing immobilized mycolic acid antigens obtained by the method of the fourth aspect of the invention.

The mycolic acid antibodies real-time inhibition test may be that of U.S. Pat. No. 7,851,166, which is hence incorporated herein by reference.

According to a sixth aspect of the invention, there is provided a method of diagnosing tuberculosis, the method including:
introducing isolated mycolic acid antigens of tuberculous mycobacterial origin onto outer surfaces of the nanoparticles to obtain mycolic acid antigen containing nanoparticles, wherein the mycolic acid antigens are presented as biomarker capturing agents;
activating a screen-printed electrode;
coating the activated screen-printed electrode with a thiolated hydrophobic substance;
dissolving mycolic acid of tuberculous mycobacterial origin in a solvent to form a mycolic acid solution;
immobilising mycolic acid antigens from the mycolic acid solution on the activated screen-printed electrode;
incubating a sample from a patient suspected of having active tuberculosis with the mycolic-acid containing nanoparticles in order to produce a control sample;
incubating a sample from the patient with nanoparticles that do not contain mycolic acid in order to produce a test sample;
contacting the control sample and the test sample with the, or an, activated screen-printed electrode containing the immobilised mycolic acid antigens in order to allow any biomarker anti-mycolic acid antibodies in each sample to bind to the immobilised mycolic acid antigens; and
using electrochemical impedance spectroscopy to measure the degree of antibody binding to the immobilised antigens in each sample,

wherein any lesser binding in the control sample compared to the test sample is a result of biomarker anti-mycolic acid antibodies in the control sample binding to the immobilised antigens and is indicative of active tuberculosis in the patient.

The nanoparticles may be of a poly(lactic-co-glycolic acid) (PLGA).

The thiolated hydrophobic substance may be octadecanethiol.

The screen-printed electrode may be a gold screen-printed electrode. The screen-printed electrode may be solvent-resistant. The screen-printed electrode may be a disposable electrode.

The activation of the screen-printed electrode may be by chemically and mechanically polishing the electrode. The chemical polishing may be effected first, whereafter the mechanical polishing is effected.

An acid may be used for the chemical polishing of the screen-printed electrode. The acid may be piranha acid. Alumina may be used for the mechanical polishing of the screen-printed electrode.

The solvent in which the mycolic acid is dissolved to form the mycolic acid solution, may be dimethylformamide.

The screen-printed electrode may be exposed to the mycolic acid solution for a period of about one hour.

According to a seventh aspect of the invention, there is provided a method of diagnosing tuberculosis, which includes
introducing isolated mycolic acid antigens of tuberculous mycobacterial origin onto particles;
activating a screen-printed electrode by both chemically and mechanically polishing it;
coating the screen-printed electrode with a thiolated hydrophobic substance;
dissolving mycolic acid of tuberculous mycobacterial origin in a solvent to form a mycolic acid solution;
immobilising mycolic acid antigens from the mycolic acid solution on the activated screen-printed electrode;
incubating a sample from a patient suspected of having active tuberculosis with the mycolic-acid containing particles in order to produce a control sample;
incubating a sample from the patient with particles that do not contain mycolic acid in order to produce a test sample;
contacting the control sample and the test sample with the, or an, activated screen-printed electrode containing the immobilised mycolic acid antigens in order to allow any biomarker anti-mycolic acid antibodies in each sample to bind to the immobilised mycolic acid antigens; and
using electrochemical impedance spectroscopy to measure the degree of antibody binding to the immobilised antigens in each sample,

wherein any lesser binding in the control sample compared to the test sample is a result of mycolic acid antibodies in the control sample binding to the immobilised antigens and is indicative of active tuberculosis in the patient.

The chemical polishing may be effected first, whereafter the mechanical polishing is effected.

An acid may be used for the chemical polishing of the screen-printed electrode. The acid may be piranha acid. Alumina may be used for the mechanical polishing of the screen-printed electrode.

The particles may be nanoparticles as hereinbefore described.

The solvent in which the mycolic acid is dissolved to form the mycolic acid solution, may be dimethylformamide.

The screen-printed electrode may be exposed to the mycolic acid solution for a period of about one hour.

The nanoparticles may be of a poly(lactic-co-glycolic acid) (PLGA).

The thiolated hydrophobic substance may be octadecanethiol.

The screen-printed electrode may be a gold screen-printed electrode.

The screen-printed electrode may be solvent-resistant. The screen-printed electrode may be a disposable electrode.

According to an eighth aspect of the invention, there is provided a method of diagnosing tuberculosis, which includes
introducing isolated mycolic acid antigens of tuberculous mycobacterial origin onto particles;
activating a screen-printed electrode;
coating the screen-printed electrode with a thiolated hydrophobic substance;
dissolving mycolic acid of tuberculous mycobaterial origin in dimethylformamide to form a mycolic acid solution;
immobilising mycolic acid antigens from the mycolic acid solution on the activated screen-printed electrode;
incubating a sample from a patient suspected of having active tuberculosis with the mycolic-acid containing particles in order to produce a control sample;
incubating a sample from the patient with particles that do not contain mycolic acid in order to produce a test sample;
contacting the control sample and the test sample with the, or an, activated screen-printed electrode containing the immobilised mycolic acid antigens in order to allow any mycolic acid antibodies in each sample to bind to the immobilised mycolic acid antigens; and
using electrochemical impedance spectroscopy to measure the degree of antibody binding to the immobilised antigens in each sample,

wherein any lesser binding in the control sample compared to the test sample is a result of mycolic acid antibodies in the control sample binding to the immobilised antigens and is indicative of active tuberculosis in the patient.

The activated screen-printed electrode may be exposed to the mycolic acid solution for a period of about one hour.

The particles may be nanoparticles as hereinbefore described.

The activation of the screen-printed electrode may be by chemically and mechanically polishing the electrode. The chemical polishing may be effected first, whereafter the mechanical polishing is effected.

An acid may be used for the chemical polishing of the screen-printed electrode. The acid may be piranha acid. Alumina may be used for the mechanical polishing of the screen-printed electrode.

The nanoparticles may be of a poly(lactic-co-glycolic acid) (PLGA).

The thiolated hydrophobic substance may be octadecanethiol.

The screen-printed electrode may be a gold screen-printed electrode.

The screen-printed electrode may be solvent-resistant. The screen-printed electrode may be a disposable electrode.

According to a ninth aspect of the invention, there is provided a point of care tuberculosis diagnostic kit, which includes a) a first control sample container containing dry mycolic acid antigen coated nanoparticles; b) a second test sample container containing the same amount or quantity of uncoated nanoparticles, and c) an individually wrapped, activated, organic solvent resistant, mycolic acid coated, screen printed electrode.

The kit may include standard equipment for measuring electro-impedance as will be known to people trained in electrochemistry, and which may include a computer controlled fluidics pump feeding into a multivalve with sample injector and connected with tubing to feed into and aspirate out from the electrode surface of the electrode clamped in a screen-printed electrode holder. The electrode may be electronically connected to a portable or desk-top potentiostat equipped with software to accumulate and interpret electrochemical signals from the electrode, calculate electro-impedance values and display the results in a Nyquist plot.

The containers may typically each be a tube.

The invention will now be described in more detail with reference to the Example hereunder, and the accompanying drawings.

In the drawings:

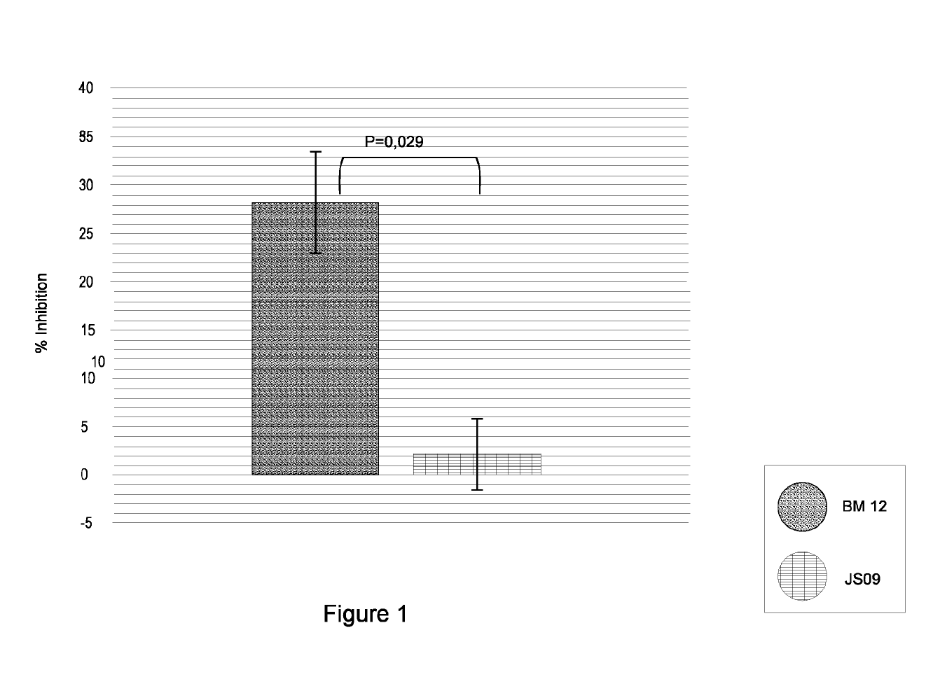

FIG. 1 shows, for the Example, the statistical analysis of percent inhibition of TB positive (BM12) and TB negative (JS09) human serum samples as determined by SPR MARTI; error bars represent standard error of mean with n=3;

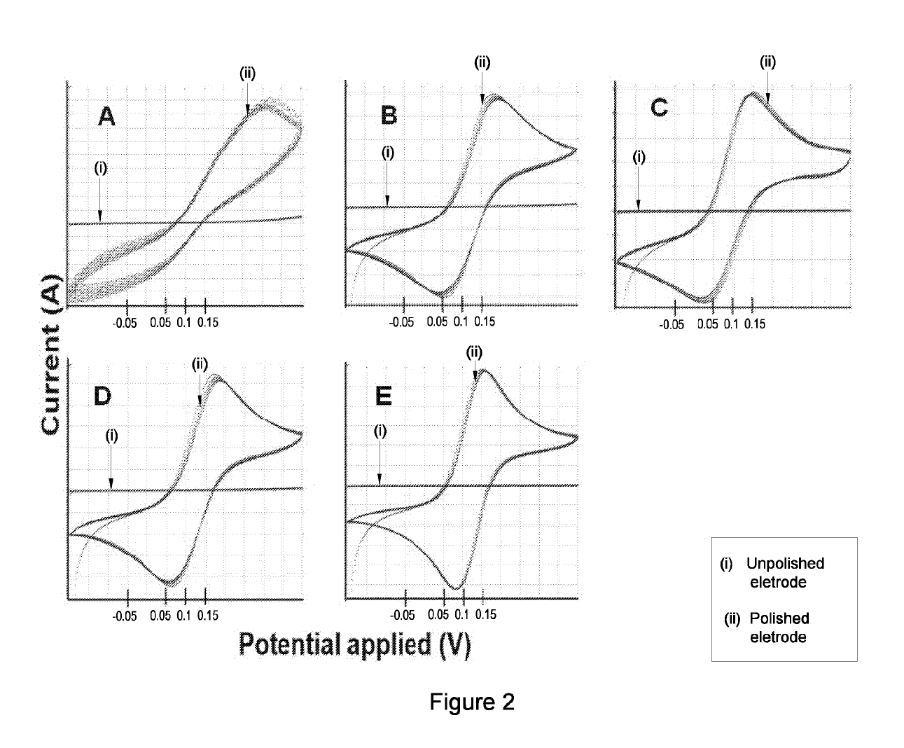

FIG. 2 shows, for the Example, cyclic voltammetry profiles of differently polished gold screen-printed electrodes: A) Electrochemical polishing with 0.5 M H₂SO₄; B) Mechanical polishing with alumina; C) Chemical polishing with piranha acid; D) Combination of electrochemical and mechanical polishing; E) Combination of chemical and mechanical polishing. Scan rate was 50 mVs⁻¹;

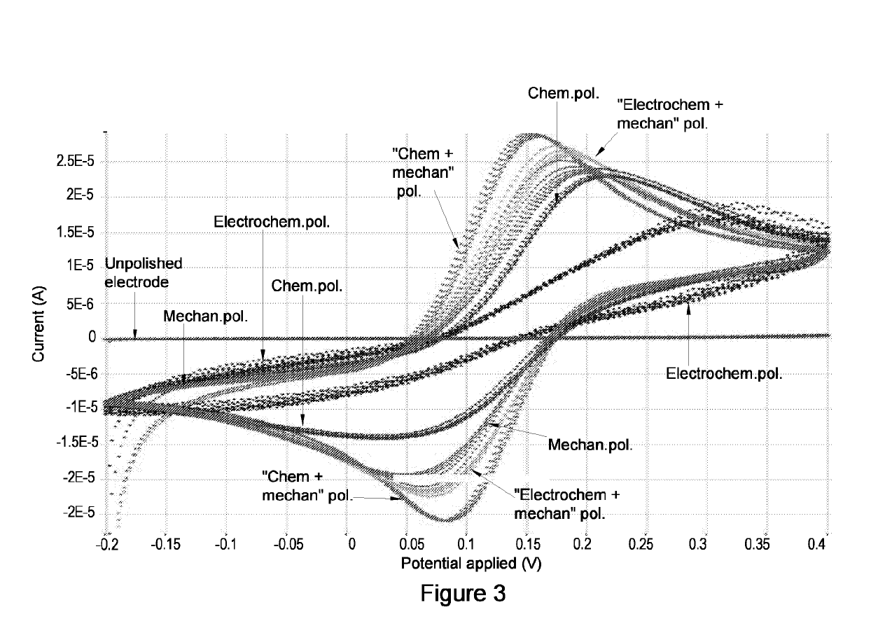

FIG. 3 shows, for the Example, cyclic voltammetry of electrode surfaces after applying different polishing methods. Scan rate was 50 mVs⁻¹;

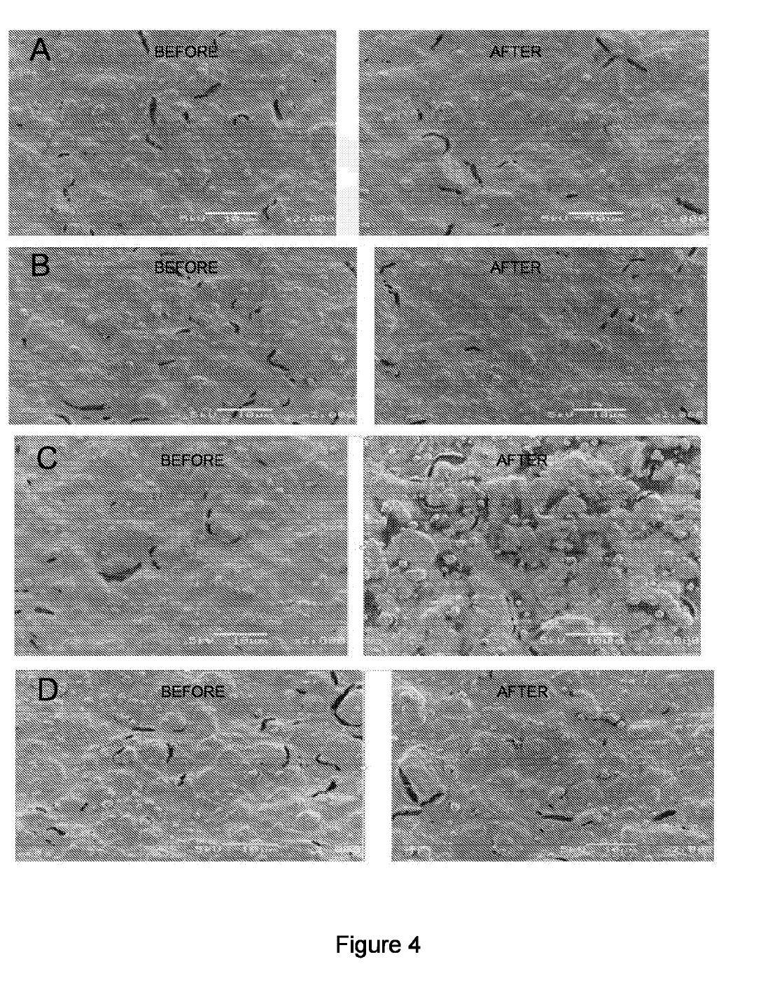

FIG. 4 shows, for the Example, scanning electron microscopic analysis of a gold electrode surface before and after polishing. A) Electrochemical and mechanical polishing; B) Chemical and mechanical polishing; C) Mechanical polishing (gently polished); D) Mechanical polishing (strongly polished);

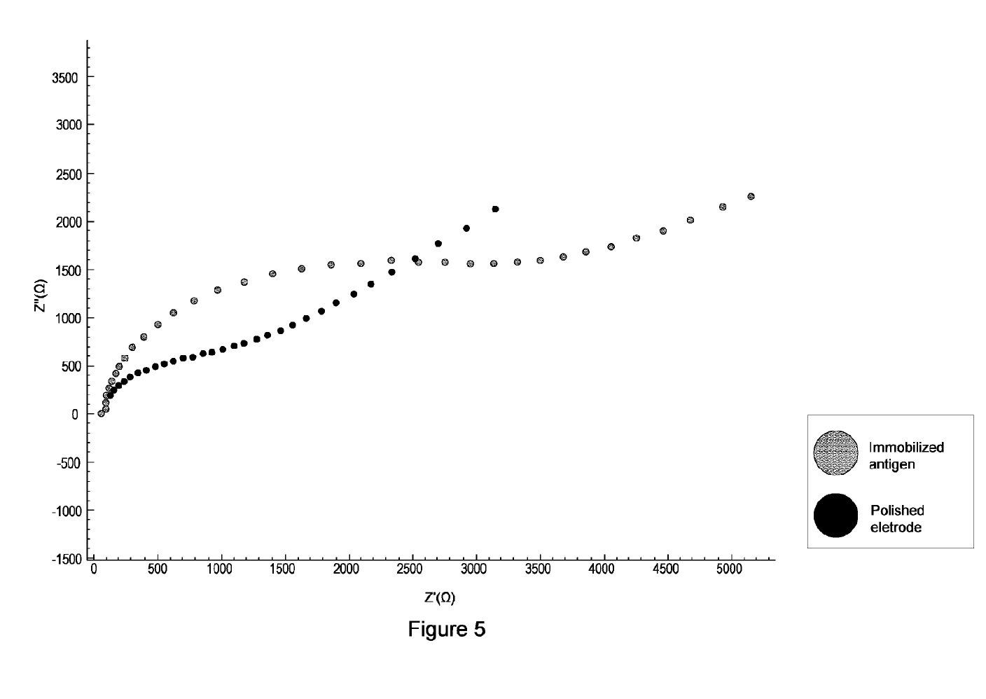

FIG. 5 shows, for the Example, a Nyquist plot of the polished electrode, before and after coating with immobilized MA antigen on an ODT coated gold SPE;

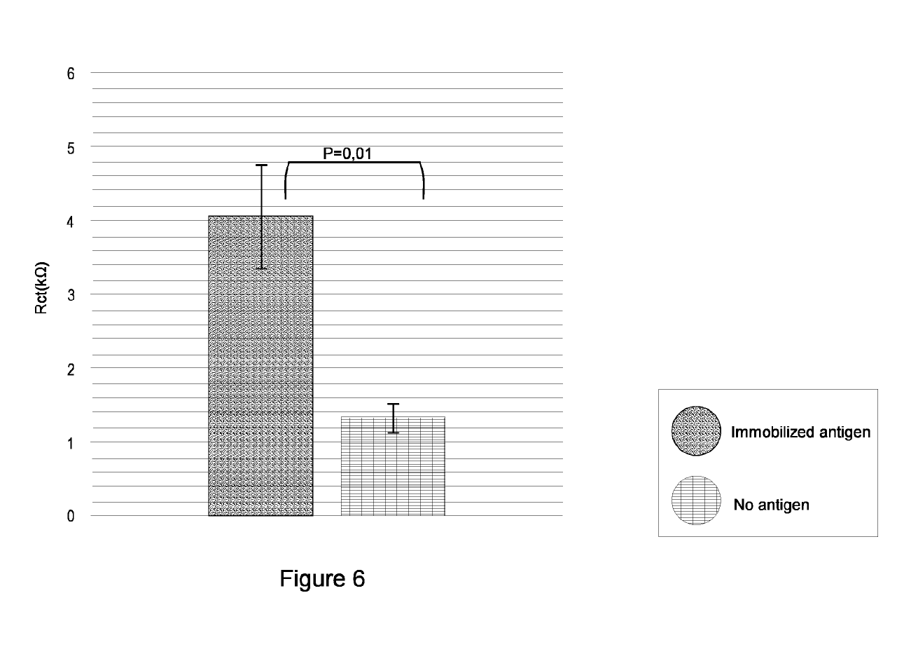

FIG. 6 shows, for the Example, a statistical analysis of Rct values from electro-impedance measurements before and after MA antigen immobilization on “combination of chemically and mechanically” polished SPEs; error bars represent a standard deviation with n=3;

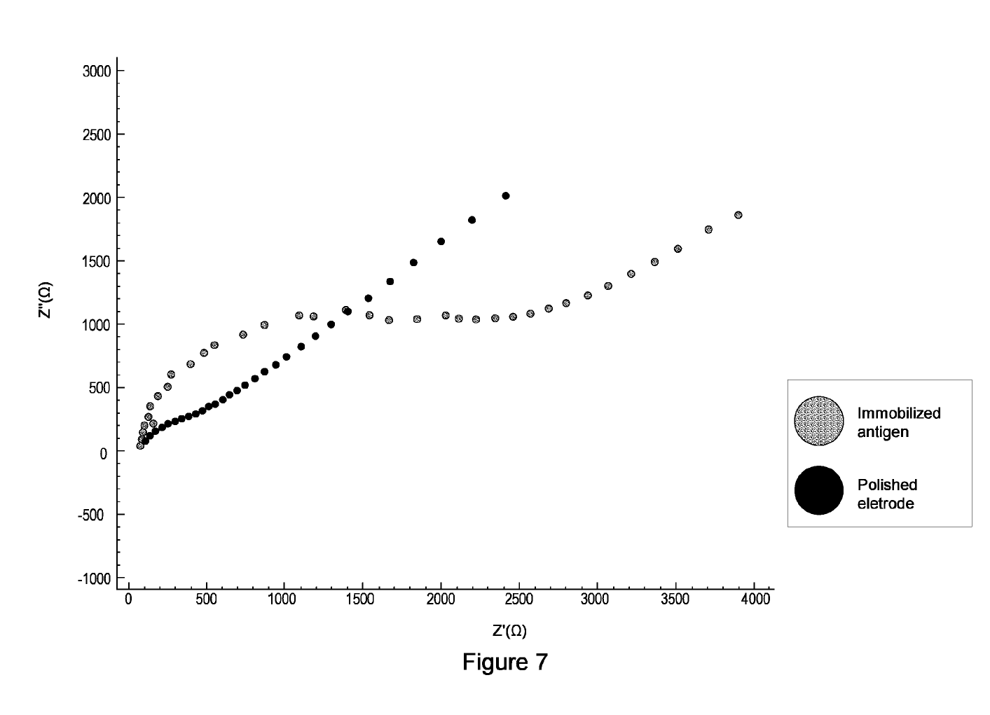

FIG. 7 shows a Nyquist plot of the polished electrode, before and after coating with immobilized MA antigen on an ODT coated gold SPE for the Example, as a demonstration of the negative effect of using expired dimethylformamide to coat MA antigen on SPEs;

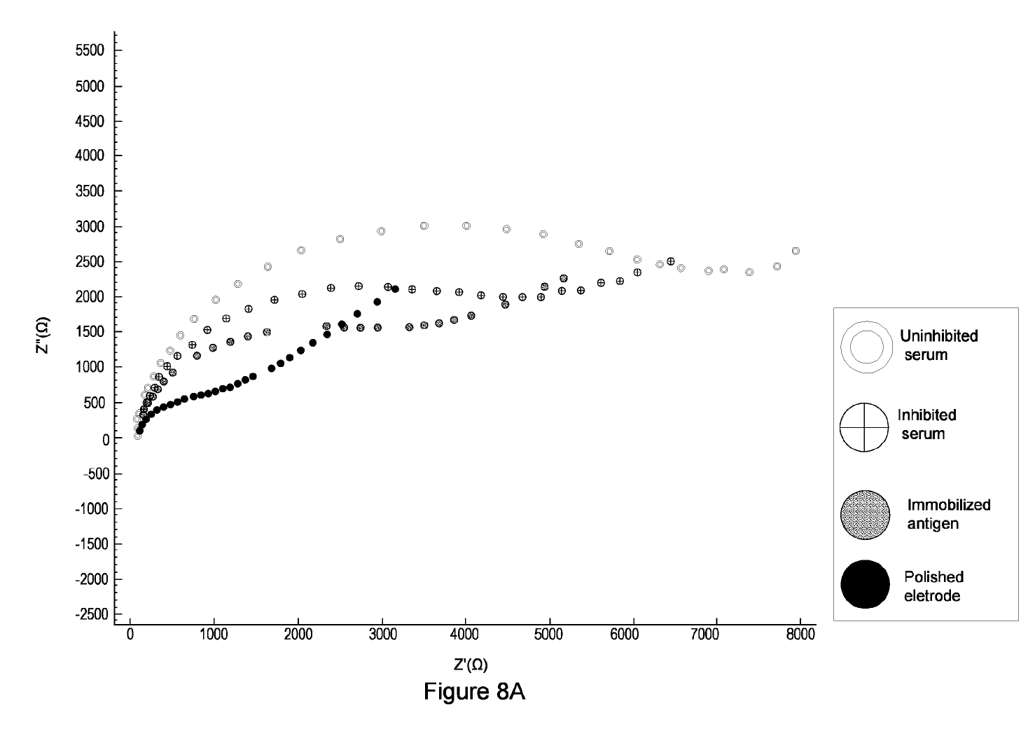

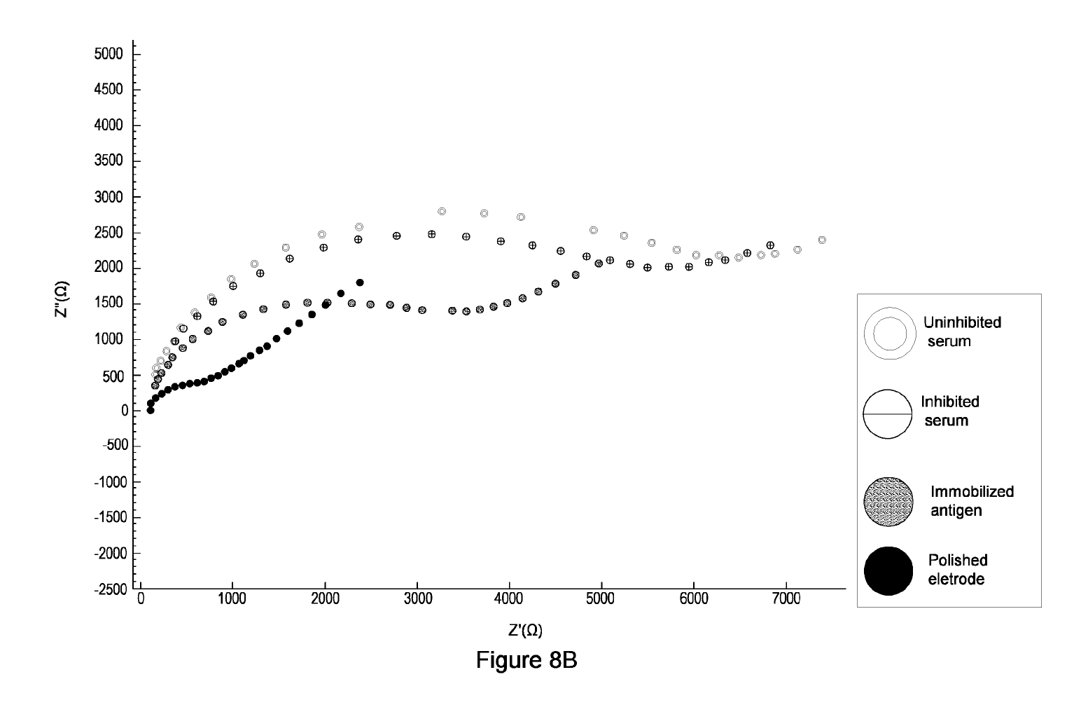

FIG. 8A shows, for the Example, a Nyquist plot outcome for the detection of anti-MA antibodies in a TB positive human serum (BM12) with the improved prototype EIS-based MARTI test;

FIG. 8B shows, for the Example, a Nyquist plot outcome for the detection of anti-MA antibodies in a TB negative human serum (JS09) with the improved prototype EIS-based MARTI test; and

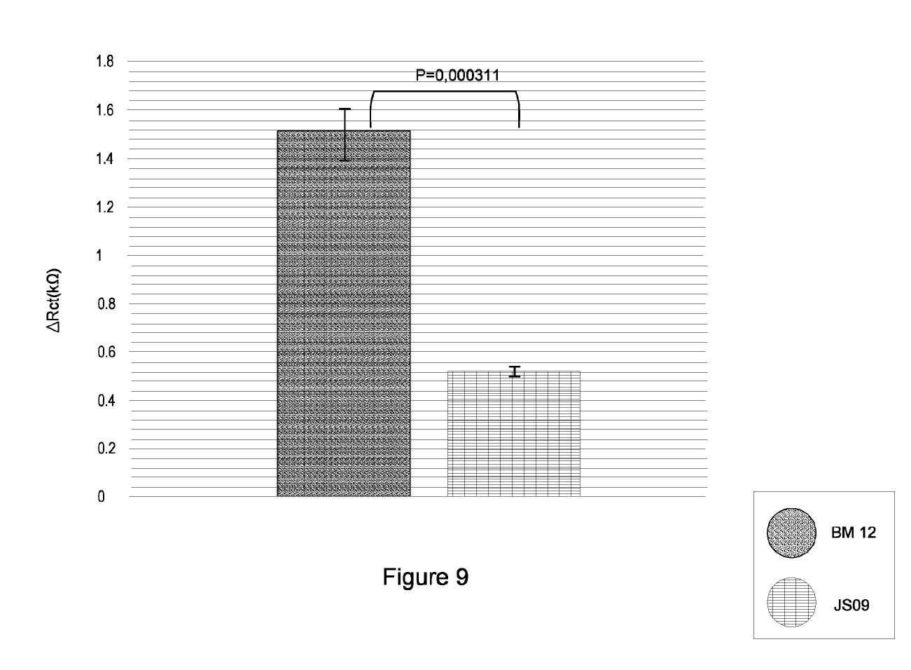

FIG. 9 shows, for the Example, the Nyquist ΔR_{ct} value differences of the prototype EIS-based MARTI test for TB positive (BM12) and TB negative (JS09) patient sera using gold SPEs; error bars represent a standard error of mean with n=5.

### EXAMPLE

Materials and Methods

Materials

Unless otherwise specified, all reagents were at least 99.5% pure and purchased from either Sigma-Aldrich or Merck. Distilled de-ionised water (ddd H₂O) from the Elga water system (Labotec, South Africa) was used for the preparation of reagents and rinsing of SPEs. Resistivity of all dddH₂O used was 18.2 MΩ.cm.

Blood samples were allowed to clot for 4 h, serum aspirated into clean 1.5 mL eppendorf tubes, centrifuged at 4° C. to eliminate red blood cells, serum (35 μL) aliquotted in 600 μL eppendorf tubes and stored at −70° C. until use.

20× Phosphate Buffered Saline

To make up the solution, 4 g KCI, 21 g Na₂HPO₄, 160 g NaCl and 4 g KH₂PO₄ were mixed with 850 mL ddd H₂O and then made up to 1 litre with ddd H₂O.

1× PBS

To make up the solution, 50 mL of 20× PBS was mixed with 850 mL of ddd H₂O (pH at 7.4), made up to 1 L with ddd H₂O and filtered through 0.2 μm cellulose acetate filters (Sartorius Stedium Biotech, Germany).

1× PBS/AE

To make up the solution, 50 mL of 20× PBS, 0.380 g Na₂EDTA and 0.250 g NaN₃ were mixed with 850 mL of ddd H₂O, adjusted to pH 7.4 with 1 M hydrochloric acid and then made up to 1 L with ddd H₂O and filtered through 0.2 μm cellulose acetate filters. Resistivity of all dddH₂O used was 18.2 MΩ.cm.

Poly(lactic-co-glycolic Acid) Nanoparticles Solution

A mass of 1 mg Poly(lactic-co-glycolic acid) nanoparticles, either with MA or without, were weighed out and suspended in 450 μL of 1× PBS/AE.

Mycolic acid (Sigma-Aldrich, South Africa) used in preparation of mycolic acid poly(lactic-co-gylcolic acid) (MA-PLGA) were sourced from Myobacterium tuberculosis (bovine strain). Sizes of MA-PLGA and PLGA particles were 259 ηm and 338 ηm respectively. Zeta potentials of MA-PLGA and PLGA were −7.61 mV and −2.5 mV respectively. The polydispersity index values determined by dynamic light scattering for MA-PLGA and PLGA were 0.241 and 0.265 respectively. Whereas monodisperse particles have a PDI of 0, PDI values ranging between 0.1 to 0.4 are regarded as moderately polydisperse.

The solvent-resistant gold SPEs used for this research were purchased from Dropsens™ (Llanera, Asturias, Spain). It consists of a gold disc-shaped working electrode, a silver pseudo electrode which served as the reference electrode and a gold counter electrode. These electrodes were screen-printed on a ceramic substrate.

Hexacyanoferrate (1 mM) (redox probe solution) (Sigma-Aldrich, South Africa) was made up in 1× PBS/AE. All electrodes used were solvent resistant gold screen printed electrodes (Dropsens™, Llanera, Asturias, Spain).

Stock Mycolic Acid Solution

To prepare a 1 mg/mL stock solution of MA in chloroform, analytical grade chloroform (180 μL) was added to a vial that contained MA (1 mg) to reconstitute previously aliquotted MA.

Octadecanethiol (ODT) Solution

ODT (0.1146 g) was weighed out and added to hexane (4 mL). The capped vial containing the ODT solution was sonicated in a Bransonic Model 42 water bath sonicator at room temperature for 5 min to allow the ODT (0.1 M) to completely dissolve in hexane.

All gold SPEs used in the research were solvent-resistant. For all cyclic voltammetry measurements and Nyquist plot analysis, a Metrohm autolab PGSTAT302N potentiostat (Utrecht, Netherlands) was used. The statistical software used was NOVA 1.8. Before any electrochemistry was carried out, the SPEs were polished either by mechanical polishing (MechanPol), a combination of electrochemical and is mechanical polishing (E+M) or a combination of chemical and mechanical polishing (C+M). The SPEs were characterized by performing five cyclic voltammetry scans per electrode in redox probe solution ([Fe(CN)₆]⁴⁻/[Fe(CN)₆]³⁻). The cycling was carried out between the range of −0.2 V to +0.4 V at a scanning rate of 50 mVs⁻¹.

Dimethylformamide (DMF) (99.8% anhydrous, Sigma-Aldrich, South Africa) was delivered in 200 mL amber injection bottles, sealed with a crimped rubber bung with a centre tear-off seal. Extreme care was taken when DMF was used, due to its toxicity and instability towards humidity and oxygen. The half-life of DMF in water is 36 h and 192 h in air. DMF was aspirated from its container upside down with a sterile needle attached to a 10 mL sterile syringe (LASEC, South Africa).

ODT Coating

Electrodes were placed in a hexane chamber and coated using the drop-dry method with 20 μL 0.1 M ODT-hexane solution for 8 min at room temperature, to allow complete solvent evaporation. Care was taken to ensure that only the area containing the bare electrode surfaces were coated. The coated electrodes were removed from the chamber and sprayed twice with absolute ethanol and allowed to dry on the work bench at room temperature, typically for no longer than 5 min.

MA-PLGA (1 mg) and PLGA (1 mg) were dissolved in separate vials of redox buffer (450 μL in each vial) and vortexed.

Standard Serum Dilution

10 μL of patient serum was diluted in 90 μL of redox probe solution and gently vortexed.

Methods

Formulation of PLGA and MA-PLGA Nanoparticles

PLGA (100 mg) was dissolved in dichloromethane (DCM) (6 mL). Mycolic acid (1.8 mg) was dissolved in DCM, vortexed and added dropwise to the PLGA-dichloromethane solution. This was followed by the addition of 2% (w/v) polyvinyl alcohol (2 mL in de-ionised water) to the MA-PLGA-dichloromethane solution. The resultant suspension was homogenised for 5 min at 20 000 revolutions per minute (rpm). The emulsion was added to 2% (w/v) polyvinyl alcohol (40 mL in de-ionised water) and homogenised for 5 min at 20 000 rpm. The emulsion was left stirring overnight in a water bath sonicator. The emulsion was centrifuged at 4000 g for 10 min at 10° C. to collect pellets as large particles. Supernatant which contained fine particles was removed and centrifuged at 21000 g for 15 min at 10° C. to collect pellets. Supernatant was discarded and pellets were dispersed in 3% trehalose (5 mL, w/v in de-ionised water) and freeze dried for four days. The sizes and zeta potential of the particles were analysed on a zetasizer Nano ZS (Malvern, UK). The same procedure was applied when PLGA alone was made except that no addition of MA was done.

Coating of SPR Gold Disc

SPR sensor unpolished gold discs (AUTOLAB, Netherlands) were placed overnight in 0.1 M ODT-absolute ethanol solution that had been sonicated for 30 min in a Bransonic Model 42 water bath sonicator at room temperature. The gold disc was washed with absolute ethanol, allowed to dry and placed on a glass prism that contained a drop of refractive index oil.

Surface Plasmon Resonance

The standard MARTI protocol for SPR (Lemmer et al. 2009) was used with minor modifications. In brief, a SPR resonance dip analysis was carried out to investigate the integrity of the sensor surface of the coated disc. A baseline using PBS/AE was set, followed by MA liposomes (50 μL) addition on the gold disc for 20 min. This was followed by PBS/AE wash and saponin treatment to prevent non-specific binding. A 1/500 diluted serum was added on the gold disc in each cell. The signals obtained in each cell were aligned on each other. Pre-incubation of 1/250 diluted serum containing either MA-PLGA (50 μL) or PLGA (50 μL) was carried out for 20 min at room temperature. Pre-incubated MA-PLGA-serum dilution (10 μL) was added in one cell, addition of PLGA-serum dilution (10 μL) in the other cell and data of binding collected. Data was transported to Microsoft Excel for evaluation and statistical analysis.

Mechanical Polishing

A drop of 0.05 μM alumina (BASi Instruments, Indiana, USA) was added onto the sensor surface of the gold SPE and the electrode was then hand polished on a polishing pad for 30 seconds in a clockwise and anti-clockwise motion. The electrode was rinsed with triple distilled water (dddH₂O) and polished a second time with alumina as detailed above. The SPE was then sonicated in a sonication water bath over a time period of 2 min, so as to rid the surfaces of any alumina traces (Yang et al. 1995). Following this procedure, the SPE was functionally characterized in a redox probe solution ([Fe(CN)₆]⁴⁻/[Fe(CN)₆]³⁻).

Combination of Electrochemical Polishing Method and Mechanical Polishing

The SPE was electrochemically pre-treated in 0.5 M sulphuric acid (H₂SO₄) and cycling was carried out between the ranges of −0.1 V to +1.2 V at a scanning rate of 100 mVs⁻¹. The cyclic voltammetry consisted of 25 CV scans, with a step potential of 0.00244 V. The electrochemically pre-treated electrode was then rinsed in dddH₂O, allowed to dry in room temperature and characterized in a redox probe solution ([Fe(CN)₆]⁴⁻/[Fe(CN)₆]³⁻). Following the characterization of the electrochemically pre-treated electrode, the SPE was rinsed with dddH₂O, allowed to dry at room temperature and then mechanically pre-treated using the procedure for mechanical polishing as detailed above.

Combination of Chemical Polishing Method and Mechanical Polishing

Chemical polishing of SPEs was performed using hot piranha acid (30% hydrogen peroxide and concentrated sulphuric acid in a 1:3 v/v ratio). Piranha acid is a highly corrosive solution, thus safety precautions had to be taken whilst using the acid. Piranha acid was freshly prepared, taking into account that the acid is exothermic. A SPE was dipped into a hot piranha acid for 10 min, rinsed thoroughly with dddH₂O, allowed to dry at room temperature and functionally characterized in a redox probe solution ([Fe(CN)₆]⁴⁻/[Fe(CN)₆]³⁻). Following the characterization of the chemically pre-treated electrode, the SPE was rinsed with dddH₂O, allowed to dry at room temperature and then mechanically pre-treated using the procedure for mechanical polishing as detailed above. The gold oxide that was formed due to chemical polishing was reduced by dipping the electrode in absolute ethanol for 1 h.

Scanning Electron Microscopy

For all microscopy work performed, a JSM-5800LV scanning microscope (Thermo Scientific) was used. A conductive tape was attached to the metal surface at the bottom of the electrode and the rest of the tape was attached to a metal plate. Two spots were viewed on the electrode at four different magnifications (500×, 2 000×, 5 000× and 10 000×). The 2 000× magnification was chosen over other magnifications to display the results obtained from microscopy because it provides an overall perspective of the gold electrode surface.

Mycolic Acid Antigen Immobilization, with DMF, on Solvent Resistant Screen Printed Electrodes

DMF (1 mL) was added to a vial that contained MA (0.5 mg), heated for 20 min, vortexed and allowed to cool down. From the resultant MA solution, 100 μL was added onto the gold portion of an ODT coated gold SPE that was placed in a petri dish and incubated for 1 h at room temperature. Adequate care was taken to ensure that the surface on which the SPE rested on was dumpy-levelled. After incubation time had elapsed, de-ionised water (50 mL) was added in the petri dish to rapidly wash away the DMF solution from the SPE. The edge of the gold SPE was blotted on a small stack of paper towel, allowed to dry at room temperature for 5 min and analysed on a potentiostat.

Antibody Inhibition Study of MA Immobilized Antigen on ODT-Coated Electrodes

Electrodes were polished according to the procedure above, coated with ODT and MA antigens were immobilized on SPEs. Redox probe containing MA-PLGA (300 μL) and PLGA (300 μL) were mixed with diluted serum (20 μL) and incubated for 10 min at room temperature. SPE was placed inside a flow cell and connected to a port. The valve position of the sample loading injector was switched to load and MA-PLGA-serum dilution (150 μL) was loaded from the sample into the flow tubes. The valve position was switched back to inject and the pump injected the MA-PLGA-serum dilution onto the sensor surface of the SPE at a flow rate of 0.05 mL/min for 10 min. A Nyquist plot analysis was performed with NOVA 1.8 software. Next, the PLGA-serum dilution (150 μL) was loaded from the sample loading outlet into the flow tubes of the potentiostat after the valve position had been switched to load. The valve position was switched back to inject and the potentiostat pump injected the PLGA-serum onto the sensor surface at a flow rate of 0.05 mL/min for 10 min. Then a Nyquist plot analysis was performed with NOVA 1.8 software.

Results and Discussion

Poly(lactic-co-glycolic Acid) Nanoparticles as a Presentation Vehicle for Anti-Mycolic Acid Biomarker (SPR Biosensor)

PLGA was investigated as a suitable substitute for liposomes as carriers for the MA inhibitor for the standardized MARTI test on a SPR biosensor (Lemmer et al. 2009). SPR analysis was performed for both a TB positive serum (BM12and TB negative serum (JS09). A student t-test was used for statistical analysis. The expected result was achieved with a clear difference between the inhibited signal and the uninhibited signal for a TB positive patient serum and no difference between the inhibited and the uninhibited signal for a TB negative patient serum. Statistical analysis (two sample t-test assuming unequal variance) was performed to determine if there was a statistical significance between TB positive serum sample (BM12) and TB negative serum sample (JS09). To achieve this, % inhibition was calculated as described below.

% Inhibition=slope difference×100%

Slope difference=slope of uninhibited serum−slope of inhibited serum

N.B. Slope of uninhibited/inhibited serum is the difference between the first exposure of pre-incubated serum (at 3650 sec) and 55 seconds after exposure (at 3705 seoc)

The summary data in FIG. 1 indicates a 28% inhibition for TB positive serum sample using nanoparticles. This is comparable to data reported by Ejoh (2014) which reported a near 27% inhibition for a TB positive serum sample (BM12) using liposomes. As shown in FIG. 1, there is a significant difference between TB positive serum sample (BM12) and TB negative serum sample (JS09) with p<0.05. SPR results using PLGA nanoparticles as an empty carrier agent showed that it does not exhibit inhibitory action of biomarker anti-MA antibodies by itself. This result supports the hypothesis that PLGA nanoparticles can be used to substitute liposomes as a carrier/presenter for the MA antigen inhibitor in the SPR based MARTI test.

Quantitative Analysis by Cyclic Voltammetry of Different Electrode Polishing Methods

The purpose of polishing solvent-resistant SPEs was to remove the solvent protective layer that was present on the sensor surface of the electrodes. Cyclic voltammetry was used to analyze polished gold SPEs for their electrochemical functionality. The cyclic voltammetry measurements consisted of 5 CV scans, with a set potential of −0.198 V and a step potential of 0.00244 V. FIG. 2 displays the cyclic voltammetry profiles for the different polishing methods that were investigated.

As shown in FIG. 2 A-E, the surfaces of the unpolished electrodes were completely blocked and inactive before any electrode polishing was done. Polishing was required to activate the sensor surface before antigen immobilization. After polishing, the expected cyclic voltammetry patterns appeared, showing measurable peak currents (iₚ) and peaks separation (ΔE) of separated oxidation and reduction peaks for each type of polishing method. The strong acids used in chemical and electrochemical polishing were expected to hydrolyse the ester bonds of the protective polyester coat that covered the electrode surfaces and therefore activate the electrode surface. It was expected that the chemical or electrochemical polishing methods would produce more reliable results of electrode activation than mechanical hand polishing, but this was found not to be the case.

The number of electrons transferred as the reaction occurred can be determined from the separation between the peak potentials. Separation between peak potential is given by the equation below:

ΔEₚ=Eₚₐ−E_{pc}=(0.059/n)V

The theoretical value for a fast one-electron transfer is ΔEₚ=59 mV. At this value, electron transfer is at its peak. Peak separation increases where the electron transfer is slow at the electrode surface (Kissinger & Heineman, 1983). For a reversible cyclic voltammetry, the peak current is given by the Randles-Sevcik equation (at 25° C.)

iₚ=(2.69×10⁵)n^{3/2}ACD^{1/2}v^{1/2}

where “n” is number of electrons, “A” is electrode area (cm²), “C” is concentration (mol/cm³), “D” is diffusion coefficient (cm²/s) and v is potential scan rate (V/s). The Randles-Sevcik equation describes the effect of scan rate on the peak current, iₚ. At a faster voltage scan rate, iₚ increases and is directly proportional to concentration. For a fast one-electron transfer with ΔEₚ=59 millivolts (mV), the values of iₚₐ and i_{pc} should be identical for a reversible system (Kissinger & Heineman, 1983).

It was expected that at least one of the polishing methods would give a peak separation of about 59 mV. Cyclic voltammetry scan repeats of electrochemically polished electrodes were rough and poor. The peak separation of electrochemically lo polished electrodes was approximately 300 mV (FIG. 2A). Mechanical polishing of gold SPEs was better than electrochemical polishing. This is based on the amplitude of oxidation and reduction peak heights of mechanically polished electrodes. Cyclic voltammetry scan repeats of mechanically polished electrodes were reproducible and smooth. Peak separation of mechanically polished electrodes was about 100 mV (FIG. 2B).

Chemical polishing (FIG. 2C), combination of “electrochemical and mechanical” polishing (FIG. 2D) and “chemical and mechanical” polishing (FIG. 2E) also improved sensor surface based on the amplitudes of their peak heights and smoothness of their CV scan repeats. Peak separation of chemically, ‘combination of electrochemically and mechanically” and ‘combination of chemically and mechanically” polished electrodes were about 120 mV, 100 mV and 75 mV respectively. A combination of chemically and mechanically polished electrodes gave the closest peak separation value (75 mV), which is close to the theoretical value of 59 mV.

All five types of polishing methods explored were overlaid in one cyclic voltammetry profile (FIG. 3). From FIG. 3, the best three polishing methods for a gold SPE were selected based on the heights of their peak current, peak separation and stability based on scan repeats. The best polishing methods were mechanical polishing (M), a combination of “electrochemical and mechanical polishing” (E+M) and a combination of “chemical and mechanical polishing” (C+M). These three polishing methods produced the highest and lowest oxidation and reduction peaks, as well as a peak separation that was closest to 59 mV.

TABLE 1
Coefficient of variation of redox peak heights of
mechanical, “electrochemical and mechanical” and
“chemical and mechanical” polishing. n = 5

| | Oxidation Peak | Reduction Peak |
|---|---|---|
| Polishing Method | Coefficient of | Coefficient of |
| | Variation (n = 5) | Variation (n = 5) |
| Mechanical (M) | 7.68% | 12.35% |
| Electrochemical and Mechanical | 2.72% | 5.51% |
| (E + M) | | |
| Chemical and Mechanical (C + M) | 1.10% | 1.02% |

The coefficient of variation for the best polishing methods were calculated for both the oxidation and reduction peak heights of each polishing method. Polishing methods were repeated on five individual electrodes, n=5. The smaller the coefficient of variation, the more stable the polishing method is. The combination of “chemical and mechanical” polishing showed the least coefficient of variation, i.e. at approximately 1%.

Scanning Electron Microscopy Analysis of Polished Electrodes

Scanning electron microscopy (SEM) analyses were done on the sensor surfaces of the SPEs treated with the three best polishing methods. This was to determine how the polishing methods altered the electrode surfaces. FIG. 4 displays the pictures of different polished SPEs. SPEs were compared before and after polishing on a scanning electron microscope. All pictures on the left of FIG. 4 are the SPEs before polishing and the pictures on the right are SPEs after polishing.

SEM analysis of the electrode surface showed no significant difference in the electrode surface before and after the different polishing had been carried out, if mechanical polishing was done gently (FIG. 4A, B, C). Cleaning of the gold electrode did not alter the morphology of the sensor surface and thus there was no damage done to the electrode surface. However, it is important to note the effect of gentle mechanical polishing versus a strong mechanical polishing (FIGS. 4C and D). A strong mechanical polishing disintegrates the sensor surfaces of the gold electrode (FIG. 4D) as seen on a scanning electron microscope. However, a gentle mechanical polishing maintains the morphology of the sensor surfaces. A structurally stable electrode surface is critical for a successful antigen immobilization. Thus gentle polishing was done when electrodes were to be mechanically polished.

Taking FIG. 4 and Table 1 into account, it was determined that a combination of “chemical and mechanical polishing” is the best polishing method for a gold SPE. It was therefore chosen as the preferred polishing method to polish gold SPEs.

MA Antigen Immobilization from DMF on Gold SPEs

Antigen immoblization of MA, using DMF as a solvent, was performed on ODT coated sensor surfaces of solvent resistant SPEs. DMF was investigated as a solvent for MA because it dissolves MA and has a slow evaporation rate. One literature report suggested 48 h for MA immobilization from DMF as a solvent on gold electrodes (Mathebula et al. 2009). However, no justification was provided for the long incubation time. A 48 h incubation is not optimal for manufacture of electrodes for diagnostics, due to the instability of the solvent when exposed to air. It was assumed that 48 h was used because DMF is commonly used as a solvent for peptide, which dissolves more readily in DMF than MA. Thus, peptides may be expected to take much longer to attach onto a prepared surface from an ideal solute-solvent association. However, MA are waxy and extremely hydrophobic in nature and are thus much less soluble in DMF.

Thus it was hypothesized that 1 h would be sufficient for MA to move from DMF and bind to a hydrophobic ODT coated sensor surface. Electrodes were polished as described above, coated with ODT and MA was immobilized onto the electrode surface for 1 h in a petri dish. The Nyquist plot was used to quantify the amount of immobilized MA antigen on ODT-coated gold SPEs. Statistical analysis was performed using two-sample t-Test assuming unequal variances.

Nyquist plot analysis of immobilized MA antigen showed an increase in charge-transfer resistance, which is an indication that binding took place on the solvent-resistant gold SPEs (FIG. 5). Statistical analysis (two-sample t-test assuming unequal variances) was performed to determine if there was a significant difference between immobilized MA-antigen on a gold SPE versus no immobilized antigen on a gold SPE. To achieve this, charge-transfer resistance (R_{ct}) was calculated from the Nyquist plots of electrodes of both MA antigen immobilization and no antigen immobilization (FIG. 5).

Analysis of immobilized MA antigen from DMF on an ODT-coated gold SPE was found to be reproducible and statistically significant with p<0.05 (FIG. 6). Immobilized MA antigen registered a binding signal of approximately three times that of the electrode without antigen. With each successive binding, the “tail length” in each Nyquist plot decreased. This is known as Warburg diffusion and it is caused by a decrease in diffusion as the layer thickness of the electrode surface increases with each successive addition.

DMF oxidises and has a half-life of five days after exposure to air. The use of DMF after its half-life has a negative effect on the immobilization of MA antigen on ODT-coated gold SPEs. As shown in FIG. 7, Nyquist plot analysis of immobilized antigen reveals instability as can be shown by the breaks in the plot. Thus it is highly recommended that only freshly distilled or hermetically sealed DMF should be used for coating of SPEs with MA.

Validation of the Improved MARTI Test with Patient Serum Samples

Three challenges had been addressed thus far in the current invention towards a feasible, point of care, EIS based, MARTI test for diagnosis of TB: the substitution of the liposomal MA carrier with stable nanoparticles for the antibody inhibition; the reliable and efficient activation of solvent resistant SPEs for antigen coating; and a reliable and economic way of standardized MA antigen coating of the activated electrodes. This has paved the way for the final assembly of a prototype MARTI assay device to be tested with a TB positive and a TB negative human patient serum to determine if the prototype MARTI assay can differentiate them convincingly. Serum samples from a TB positive patient (BM12) and a TB negative human individual (JS09) were pre-incubated with PLGA nanoparticles (PLGA-NP) and MA-PLGA-NP and the anti-MA biomarker antibodies detected with EIS on two newly activated and MA antigen coated SPEs. It had been reported that the use of saponin as a blocking agent on MA antigen coated SPEs gave a large change of Ra that drastically increased resistance to charge transfer and prevented the detection of a difference between TB positive and TB negative serum (Baumeister, 2012). Thus saponin blocking was omitted from the EIS experiments.

Nyquist plot analysis was used to determine binding of antibodies to the immobilized antigens. Statistical analysis (two-sample t-test assuming unequal variances) was used to determine statistical significance of the difference of signals between TB positive and TB negative outcomes (FIGS. 8A and 8B).

R_{ct} serves as the signal of analysis in EIS results because it provides information on the binding of antibodies to the immobilized MA antigen on the gold SPE. R_{ct} is manually obtained by selecting data points from the semi-circle portion of the Nyquist plot. The computer software (NOVA) automatically extrapolates the semi-circle until it intercepts the x-axis to generate the R_{ct} value. For a TB positive profile, a clear difference between the Nyquist plot for inhibited serum and uninhibited serum was expected and this was achieved (FIG. 8A). For a TB negative profile, little difference between the Nyquist plot for inhibited serum and uninhibited serum was expected and this was manifested as such (FIG. 8B).

The Nyquist plot showed that PLGA-NP pre-incubation of serum samples does not hinder the data acquisition to determine difference in profiles between TB positive and TB negative human serum samples. As indicated in FIGS. 8A and 8B and Table 2, there is an increase in Rd with each successive step of the MARTI test, but a bigger difference between inhibited and uninhibited serum when analysing a TB positive human serum samples (ΔR_{ct}=1.789) compared to a TB negative serum (ΔR_{ct}=0.515). The ΔR_{ct} value is an indication of antibodies binding to antigen on the electrode surface. In MARTI test analyses on SPR biosensor, percent inhibition of antibody binding to the sensor surface immobilized MA between MA-antigen inhibited and uninhibited serum dilutions was used as the signal outcome that determined a TB positive or negative diagnosis (Lemmer et al. 2009).

For EIS based MARTI, however, the absolute R_{ct} value differences (ΔR_{ct}) between inhibited and uninhibited serum dilutions are used because of the inhomogeneities of the electrode surface caused by the roughness of the electrode surface during manufacture. These inhomogeneities affect the solution bulk resistance (Barsoukov & MacDonald, 2005) and thus explain the varying ΔR_{ct} values for the same lo uninhibited serum sample across different electrodes. This went as low as 2.307 and as high as 6.373 for the TB negative sample (Table 2), even though the ΔR_{ct} varied by only 0.034 when comparing the data between the inhibited and uninhibited serum sample for the same electrodes.

The larger ΔR_{ct} value for the TB positive patient compared to that of the TB negative individual is the important MARTI-outcome that should be tested for statistical significance. If significant, the prototype may be regarded as functional. The test with the TB positive and TB negative human serum samples were therefore repeated five times on five different electrodes for each sample type. From the data in Table 2, statistical analysis was performed to determine if the improved prototype MARTI test can convincingly discriminate a TB positive human serum from a TB negative human serum. The summary data in FIG. 9 indicate a near difference of 1 kΩ in signal between TB positive (1.511±0.222) and TB negative serum (0.528±0.039). Statistical significance of the approximately three fold difference in ΔR_{ct} values of TB positive and TB negative sera can be observed with P<0.0005. From this, the conclusion can confidently be made that the EIS MARTI device prototype is functional and ready for use in a validation test.

TABLE 2
Reproducibility of the prototype EIS based MARTI test to distinguish between a TB
positive and a TB negative human serum sample.

| | TB positive serum (BM12) | | | TB negative serum (JS09) | | |
|---|---|---|---|---|---|---|
| | | | ΔR_{ct} | | | ΔR_{ct} |
| | Uninhibited | Inhibited | (Uninhibited | Uninhibited | Inhibited | (Uninhibited |
| | serum (R_{ct}, | serum | Ser. − | serum (R_{ct}, | serum | Ser. − |
| Electrodes | kΩ) | (R_{ct}, kΩ) | Inhibited Ser.) | kΩ) | (R_{ct}, kΩ) | Inhibited Ser.) |
| 1 | 6.969 | 5.180 | 1.789 | 4.100 | 3.555 | 0.545 |
| 2 | 4.306 | 2.868 | 1.438 | 2.307 | 1.826 | 0.481 |
| 3 | 8.295 | 6.881 | 1.414 | 3.348 | 2.834 | 0.514 |
| 4 | 5.209 | 3.975 | 1.234 | 3.903 | 3.317 | 0.586 |
| 5 | 6.547 | 4.865 | 1.682 | 6.373 | 5.858 | 0.515 |

| | | |
|---|---|---|
| Average (R_{ct}) | 1.511 | 0.528 |
| Standard deviation | 0.222 | 0.039 |

Discussion

PLGA Nanoparticles as a Presentation Vehicle for Anti-MA Biomarker (SPR Biosensor)

The MARTI test for TB diagnosis as described by Lemmer et al. (2009) makes use of liposomes as a carrier system for MA, however liposomes suffer from many disadvantages such as weak stability over time, oxidation of the phospholipid components on exposure to air and the availability of a sophisticated tip sonicator to prepare the liposomes fresh before each use. Liposomes are usually stabilized by cholesterol, but this has to be avoided in MARTI due to cross reactivity of patient anti-MA antibodies with cholesterol and the cross-reactivity of ubiquitously present anti-cholesterol antibodies with MA (Benadie et al. 2008). These limitations previously demonstrated with the SPR-based MARTI test (Lemmer et al. 2009) are equally applicable for the EIS-based MARTI test and stand in the way of applying the test as a point of care diagnostics device.

Liposomes that were previously used with the MARTI test were 1 μm in size and did not contain cholesterol. More recently, sterol modified lipids (SMLs), in particular phosphatidylcholine, were used to stabilize the liposomes for this application without affecting the outcome of the MARTI test negatively. In SMLs, the cholesterol moieties are imbedded covalently in the acyl chains of the phospholipids (Baumeister, 2012). The presence of free cholesterol in phosphatidylcholine (PC) liposomes reduces the hydrophile-lipophile balance. Hydrophile-lipophile balance is a function of the size of the hydrophilic moieties and the strength of interaction between the lipophilic moieties of a molecule. Reduction of hydrophile-lipophile balance leads to a decrease in the curved surfaces of the liposomes, in other words, larger sizes. Addition of MA to liposomes has been shown to reduce the average size of PC liposomes (Baumeister, 2012). Of the four sterol modified lipids (PChcPC, PChemsPC, OChemsPC and DChemsPC) that had been studied, 1-palmitoyl-2-cholesterylcarbonyl-sn-glycero-3-phosphocholine (PChcPC) was shown to present the MA in the most antigenic way, a property that correlated with its tendency to shrink in size when MA was added. This suggests that a more curved surface of the liposomes, makes the MA that they carry more antigenic for the anti-MA antibodies that are prevalent as active TB biomarkers in TB positive patients.

Here, it is shown that liposomes could be replaced with PLGA nanoparticles as a more affordable, efficient and stable MA antigen presenting particle for the antibody inhibition step of MARTI. The small (MA)-PLGA particles (0.2 μm) were obtained by high g-force centrifugation. In previous experiments, filtration of nanoparticles were required to obtain useful results on SPR. Thus one may confidently anticipate that the smaller nanoparticle sizes may be preferred for better antigenicity as was found by Baumeister (2012) using the SML-liposomes. PLGA nanoparticles are better lo suited as an effective carrier system for MA than liposomes because liposomes oxidize 16 h after preparation but PLGA-NP have been reported to remain stable even after 3 months (Holzer et al. 2009).

Analysis of Electrode Polishing Method for the MARTI Test

The use of non-solvent resistant gold SPEs for the MARTI test is unable to distinguish a TB positive patient serum from a TB negative patient serum (Baumeister, 2012). This was due to organic solvents that peeled off the insulating layer of the electrodes and contaminated the sensor surfaces in the process. Using solvent resistant SPEs, it was noticed that there was a need for polishing because the solvent resistant material that covered the sensor surface needed to be removed. This led Baumeister to attempt gentle mechanical polishing on solvent resistant gold SPEs. Although better results were obtained after mechanical polishing of SPEs, quite a number of electrodes were discarded in the process (Baumeister, 2012). Thus the process of polishing needed to be standardized.

A combination of “chemical and mechanical” polishing was found to be the best suited method that can be used to get rid of the solvent resistant membrane on the sensor surface. Piranha acid, used to chemically polish the electrode, removed all polyester organic contaminant from the surface of the gold electrode. It is known that a gold oxide layer forms following piranha acid polishing of gold. However, the use of piranha acid as a chemical polisher for gold SPEs effectively cleans the electrode surface and enhances electro-catalytic activities. The presence of gold oxide adversely affects the formation of a self-assembled monolayer (SAM). Gold oxides, which are potent oxidants, are highly unstable and can be reduced by dipping the electrode in absolute ethanol.

Mechanical polishing of chemically polished SPEs further bolstered the amplitude of redox peak heights. Mechanical polishing of electrodes using alumina is one of the most common polishing methods widely used in electrochemistry. Alumina, aluminium (III) oxide, is a chemical compound that is commonly used to produce aluminium metals as a result of its hardness and stability. Mechanical polishing of a SPE with alumina greatly enhanced the surface area of the gold electrode and lo further reduced gold oxides that were present even after ethanol dipping. SEM analysis of the surface of the gold SPE following combination of “chemical and mechanical” polishing showed that there was little damage done to the electrode surface. Based on the cyclic voltammetry result obtained, following combination of “chemical and mechanical” polishing, there was a high peak current in the cathodic and anodic peaks, as well as a peak separation of about 60 mV.

Subramanian & Lakshminarayanan (1999) reported the effect of mechanical polishing by using different particle sizes of alumina (1 μm, 0.3 μm and 0.05 μm). Using Scanning Tunnelling Microscopy, they discovered that as the particle size of alumina decreased, the surface appeared smoother from a bird's eye view of the surface. SEM analysis of mechanically pre-treated electrodes using 0.05 μm alumina showed that there was little damage to the gold SPE if the electrode was gently polished.

Limited information on solvent resistant SPEs is available in the public domain, and there is no published literature in the polishing of solvent resistant SPEs. To date, there is therefore no published work that demonstrates a standardized method of polishing solvent resistant gold SPEs. The theoretical optimum reduction/oxidation peak separation of 59 mV was approximated for a combination of chemically and mechanically polished SPEs, thereby improving the standard deviation to approximately 1%. A clean and activated gold surface is of utmost importance for antigen immobilization.

MA Antigen Immobilization on ODT Coated SPEs

MA antigen immobilization on pre-polished, solvent resistant, gold SPEs was achieved by adsorption of MA to the activated SPE from a DMF solution within 1 h. Mathebula et al. (2009) used DMF as a solvent for MA to adsorb MA onto a gold electrode for detection of antibodies. Literature works reported approximately 10 h incubation period for adsorption of protein in DMF on to a solid surface (Liu et al. 2004; Xu et al. 2006) and 42 h incubation period for adsorption of protein in PBS to an electrode surface (Ciobanu et al. 2011). Liu et al. (2004) reported the adsorption of heme-proteins from DMF onto pyrolytic graphite electrodes. It is unclear why Mathebula et al. (2009) chose 48 h as an incubation time for MA adsorption from DMF on a gold electrode.

The current invention has determined that a 1 h incubation period for the adsorption of MA from DMF on to ODT-coated solvent resistant SPEs is sufficient and optimal for successful antigen immobilization.

The Prototype EIS-Based MARTI Test for TB Diagnosis

Competition immunoassays such as MARTI provide a reliable and sensitive biological assay that measures antibody responses against a range of antigens both in humans and animals (Li et al. 2001). Competitive immunoassay is mostly used when binding of antibodies are complicated by cross-reactivity and when two antibodies cannot be bound on a single molecule. A measure of the inhibited reactant provides information on the degree of inhibition. The degree of inhibition is an indication of the activity of the unknown. In the MARTI assay, ubiquitous anti-cholesterol antibodies that cross react with MA are diluted out and this enables MA antigens to be efficiently detected by antibodies by means of a competitive binding inhibition immunoassay.

Mathebula et al. (2009) and Ozoemena et al. (2010) first provided proof of principle of anti-MA antibody detection using principles of electrochemical impedance. However, their method included a washing step after serum incubation and this could reduce the detection of low affinity anti-MA antibodies that may be present in active TB patients. Baumeister (2012) demonstrated the feasibility of TB diagnosis by applying the MARTI test with EIS on a disposable electrode. However, the EIS based MARTI test still suffered from some hindrances towards a reliable point of care diagnostic, which included standardization, reproducibility, liposome stabilization and solvent compatibility for MA antigen immobilization.

The current invention was unexpectedly found to provide the three core elements that remained lacking to advance the work of Baumeister (2012) into a feasible point of care EIS-based MARTI TB diagnostic. These core elements are (1) activation of solvent resistant SPEs, (2) standardization of antigen immobilization on the biosensor surface and (3) providing a stable suspended particle for MA presentation for the antibody inhibition step. Having overcome all these hurdles, the test is now ready for validation and eventual clinical trials.

The WHO requires that an ideal point-of-care diagnostic test should satisfy the ASSURED criteria (Peeling et al. 2006). The acronym stands for (A)affordable, (S)sensitive, (S)specific, (U)user-friendly, (R)rapid and robust, (E)equipment-free and (D)deliverable. This requires that a diagnostic test should require no heavy instrumentation, be easy to use and should preferably be disposable. Another desirable property of an ideal point of care diagnostics test that can be added to the ASSURED criteria is “Not affected by HIV co-infection”. Thus an ideal TB-diagnostics test should rather satisfy the acronym, ASSURED-N. Using the methods applied in the current invention, the improved MARTI test has the potential to fulfil the ASSURED-N criteria of a TB point of care diagnostics test.

### REFERENCES

Barsoukov E, Macdonald J R (2005). Impedance spectroscopy: theory, experiment, and applications (2^{nd} edition). John Wiley & Sons Inc. (USA).

Baumeister C R (2012). Electrochemical impedance spectroscopy and surface plasmon resonance for diagnostic antibody detection. MSc research. University of Pretoria, Pretoria.

Benadie Y, Deysel M, Siko D G R, Roberts V V, Van Wyngaardt S, Thanyani S T, Sekanka G, Ten Bokum A M C, Collett L A, Grooten J, Baird M S, Verschoor J A (2008). Cholesteroid nature of free mycolic acids from M. tuberculosis. Chemistry and Physics of Lipids 152: 95-103.

Ciobanu C, Gradinaru LM, Gradinaru RV, Drobota M, Vlad S (2011). Bovine serum albumin adsorption onto UV-activated green polyurethane surface. Digest Journal of

Ejoh V E (2014). Requirements for accurate biosensor detction of anti-lipid biomarker antibodies in active TB. MSc research. University of Pretoria, Pretoria.

Holzer M, Vogel V, Mantele W, Schwartz D, Haase W, Langer K (2009). Physico-chemical characterisation of PLGA nanoparticles after freeze-drying and storage. European Journal of Pharmaceutics and Biopharmaceutics 72: 428-437.

Kissinger P T, Heineman W R (1983). Cyclic voltammetry. Journal of Chemical Education 60: 702-706.

Lawn S D, Kerkhoff A D, Vogt M, Wood R (2012). Diagnostic accuracy of a low-cost, urine antigen, point-of-care screening assay for HIV-associated pulmonary tuberculosis before antiretroviral therapy: a descriptive study. The Lancet Infectious Diseases 12: 201-209.

Lemmer Y, Thanyani S T, Very P J, Driver CHS, Venter L, Van Wyngaardt S, Ten Bokum A M C, Ozoemena K I, Pilcher L A, Fernig D G, Stoltz A C, Swai H S, Verschoor J A (2009). Chapter 5: Detection of antimycolic acid antibodies by liposomal biosensors. Methods in Enzymology 464: 79-104.

Li H, McGuire T C, Muller-Doblies U U, Crawford T B (2001). A simpler, more sensitive competition enzyme-linked immunosorbent assay for detection of antibody to malignant catarrhal fever viruses. Journal of Veterinary Diagnostic Investigation 13: 361-364.

Liu H H, Tian Z Q, Lu Z X, Zhang Z L, Zhang M, Pang D W (2004). Direct lo electrochemistry and electrocatalysis of heme-proteins entrapped in agarose hydrogel films. Biosensors and Bioelectronics 20: 294-304.

Mathebula N S, Pillay J, Toschi G, Verschoor J A, Ozoemena K I (2009). Recognition of anti-mycolic acid antibody at self-assembled mycolic acid antigens on a gold electrode: a potential impedimetric immunosensing platform for active tuberculosis. Chemical Communications 23: 3345-3347.

Ozoemena K I, Mathebula N S, Pillay J, Toschi G, Verschoor J A (2010). Electron transfer dynamics across self-assembled N-(2-mercaptoethyl) octadecanamide/mycolic acid layers: impedimetric insights into the structural integrity and interaction with anti-mycolic acid antibodies. Physical Chemistry Chemical Physics 12: 345-357.

Peeling R W, Holmes K K, Mabey D, Ronald A (2006). Rapid tests for sexually transmitted infections (STIs): the way forward. Sexually Transmitted Infections 82 Suppl 5: v1-v6.

Subramanian R, Lakshminarayanan V (1999). Effect of surface roughness on the self-assembly of octadecanethiol monolayer onto polycrystalline noble metal surfaces. Current Science 76: 665-669.

Xu Y, Wang F, Chen X, Hu S (2006). Direct electrochemistry and electrocatalysis of heme-protein based on N,N-dimethylformamide film electrode. Talanta 70: 651-655.

1. A method of making an antibody biomarker capturing vehicle suitable for use in diagnosing tuberculosis, the method including:
introducing isolated mycolic acid antigens of tuberculous mycobacterial origin onto an outer surface of a nanoparticle to obtain a mycolic acid antigen-containing nanoparticle, wherein the mycolic acid antigens are presented as antibody biomarker capturing agents.

2. The method of claim 1, wherein the nanoparticle has a size of 0.2 μm or less.

3. The method of claim 1 or 2, wherein the nanoparticle comprises poly(lactic-co-glycolic) acid.

4. An antibody biomarker capturing vehicle suitable for use in diagnosing tuberculosis, the antibody biomarker capturing vehicle comprising a nanoparticle having isolated mycolic acid of tuberculous mycobacterial origin present as a biomarker capturing agent on an outer surface thereof.

5. The antibody biomarker capturing vehicle of claim 4, wherein the nanoparticle has a size of 0.2 μm or less.

6. The antibody biomarker capturing vehicle of claim 4 or 5, wherein the nanoparticle comprises poly(lactic-co-glycolic) acid.

7. A method of activating a solvent resistant screen-printed electrode, suitable for use in diagnosing tuberculosis, the method including both chemically and mechanically polishing the electrode, thereby to obtain an activated solvent resistant screen-printed electrode.

8. The method of claim 7, wherein the chemical polishing is effected first, whereafter the mechanical polishing is effected.

9. The method of claim 7 or claim 8, wherein the electrode is a gold, solvent resistant, screen-printed electrode.

10. The method of any one of claims 7 to 9, wherein the electrode is a disposable electrode.

11. The method any one of claims 7 to 10, wherein piranha acid is used for the chemical polishing of the electrode.

12. The method of any one of claims 7 to 11, wherein alumina is used for the mechanical polishing of the electrode.

13. An activated screen-printed electrode when activated by the method of any one of claims 7 to 12.

14. A method of immobilising mycolic acid antigens on an activated screen-printed electrode surface suitable for use in diagnosing tuberculosis, the method including:
dissolving mycolic acid of tuberculous mycobacterial origin in dimethylformamide to produce a mycolic acid-dimethylformamide solution; and
incubating an activated screen-printed electrode with the mycolic acid-dimethylformamide solution to allow mycolic acid antigens to adsorb onto the activated electrode, to produce an activated screen-printed electrode containing immobilised mycolic acid antigens.

15. The method of claim 14, wherein the activated screen-printed electrode is that of claim 13 and, optionally, wherein the electrode is washed following incubation with the solution.

16. A method of diagnosing tuberculosis, the method including:
using a mycolic acid antibodies real-time inhibition test, employing electrochemical impedance spectroscopy, to diagnose the presence of active tuberculosis in a sample from a patient suspected of having active tuberculosis;
using the antibody biomarker capturing vehicle of any one of claims 4 to 6; and/or
using the activated solvent resistant screen-printed electrode of claim 13; and/or
using the screen-printed electrode containing immobilised mycolic acid antigens obtained by the method of claim 14 or claim 15.

17. The method of claim 16 wherein the mycolic acid antibodies real-time inhibition test is that of U.S. Pat. No. 7,851,166.

18. A method of diagnosing tuberculosis, the method including:
introducing isolated mycolic acid antigens of tuberculous mycobacterial origin onto outer surfaces of nanoparticles to obtain mycolic acid antigen-containing nanoparticles, wherein the mycolic acid antigens are presented as antibody biomarker capture agents;
activating a screen-printed electrode;
coating the activated electrode with a thiolated hydrophobic substance;
dissolving mycolic acid of tuberculous mycobacterial origin in a solvent to form a mycolic acid solution;
immobilising mycolic acid antigens from the mycolic acid solution on the activated electrode;
incubating a sample from a patient suspected of having active tuberculosis with the mycolic acid antigen-containing nanoparticles in order to produce a control sample;
incubating a sample from the patient with nanoparticles that do not contain mycolic acid in order to produce a test sample;
contacting the control sample and the test sample with the, or an, activated screen-printed electrode containing immobilised mycolic acid antigens in order to allow any biomarker anti-mycolic acid antibodies in each sample to bind to the immobilised mycolic acid antigens; and
using electrochemical impedance spectroscopy to measure the degree of antibody binding to the immobilised antigens in each sample,
wherein any lesser binding in the control sample compared to the test sample is a result of biomarker anti-mycolic acid antibodies in the control sample binding to the immobilised antigens and is indicative of active tuberculosis in the patient.

19. The method of claim 18, wherein the nanoparticles have a size of 0.2 μm or less.

20. The method of claim 18 or 19, wherein the nanoparticles comprise poly(lactic-co-glycolic) acid.

21. The method of any one of claims 18 to 20, wherein the thiolated hydrophobic substance is octadecanethiol.

22. The method of any one of claims 18 to 21, wherein the electrode is a gold solvent-resistant, screen-printed electrode.

23. The method of any one of claims 18 to 22, wherein the electrode is a disposable electrode.

24. The method any one of claims 18 to 23, wherein the activation of the electrode is by means of chemical and/or mechanical polishing thereof.

25. The method of claim 24, wherein chemical polishing is used, with the chemical polishing being by means of piranha acid.

26. The method of claim 24 or claim 25, wherein mechanical polishing is used, with the mechanical polishing being by means of alumina.

27. The method of any of claims 24 to 26, wherein the electrode is first chemically polished and thereafter mechanically polished.

28. The method of any one of claims 19 to 27, wherein the electrode is washed following immobilisation of the mycolic acid antigens.

29. A method of diagnosing tuberculosis, which includes
introducing isolated mycolic acid antigens of tuberculous mycobacterial origin onto particles;
activating a screen-printed electrode by both chemically and mechanically polishing it;
coating the screen-printed electrode with a thiolated hydrophobic substance; dissolving mycolic acid of tuberculous mycobacterial origin in a solvent to form a mycolic acid solution;
immobilising mycolic acid antigens from the mycolic acid solution on the activated screen-printed electrode;
incubating a sample from a patient suspected of having active tuberculosis with the mycolic-acid containing particles in order to produce a control sample;
incubating a sample from the patient with particles that do not contain mycolic acid in order to produce a test sample;
contacting the control sample and the test sample with the, or an, activated screen-printed electrode containing the immobilised mycolic acid antigens in order to allow any biomarker anti-mycolic acid antibodies in each sample to bind to the immobilised mycolic acid antigens; and
using electrochemical impedance spectroscopy to measure the degree of antibody binding to the immobilised antigens in each sample,
wherein any lesser binding in the control sample compared to the test sample is a result of mycolic acid antibodies in the control sample binding to the immobilised antigens and is indicative of active tuberculosis in the patient.

30. A method of diagnosing tuberculosis, which includes
introducing isolated mycolic acid antigens of tuberculous mycobacterial origin onto particles;
activating a screen-printed electrode;
coating the screen-printed electrode with a thiolated hydrophobic substance;
dissolving mycolic acid of tuberculous mycobaterial origin in dimethylformamide to form a mycolic acid solution;
immobilising mycolic acid antigens from the mycolic acid solution on the activated screen-printed electrode;
incubating a sample from a patient suspected of having active tuberculosis with the mycolic-acid containing particles in order to produce a control sample;
incubating a sample from the patient with particles that do not contain mycolic acid in order to produce a test sample;
contacting the control sample and the test sample with the, or an, activated screen-printed electrode containing the immobilised mycolic acid antigens in order to allow any mycolic acid antibodies in each sample to bind to the immobilised mycolic acid antigens; and
using electrochemical impedance spectroscopy to measure the degree of antibody binding to the immobilised antigens in each sample,
wherein any lesser binding in the control sample compared to the test sample is a result of mycolic acid antibodies in the control sample binding to the immobilised antigens and is indicative of active tuberculosis in the patient.

31. A point of care tuberculosis diagnostic kit for use with electrochemical impedance spectroscopy, the kit including:
a first control sample container containing dry mycolic acid antigen coated nanoparticles;
a second test sample container containing an equal quantity of uncoated nanoparticles; and
an individually wrapped, activated, solvent resistant, mycolic acid coated screen printed electrode.
